(19) United States
(12) Patent Application Publication
(10) Pub. No.: US 2023/0087961 A1
(43) Pub. Date: Mar. 23, 2023

(54) METHOD OF ENRICHING CANNABIS OIL WITH CANNABINOL

(71) Applicant: Bright Green Corporation, Wilmington, DE (US)

(72) Inventor: V. Hari, Orlando, FL (US)

(21) Appl. No.: 17/477,650

(22) Filed: Sep. 17, 2021

(51) Int. Cl. A61K 31/352; A61K 36/185; A61K 31/198; A61K 36/67

(52) U.S. Cl. CPC A61K 31/352; A61K 31/198; A61K 36/67; A61K 36/185; A61K 2236/15; A61K 2236/17; A61K 2236/37; A61K 2236/53; A61K 2236/331; A61K 2236/333

(57) ABSTRACT

A method of increasing CBN content in a Cannabinoid oil includes processing plant parts from one or more cannabis plants to create processed plant parts, extracting a Cannabinoid oil from the processed plant parts, and refining the Cannabinoid oil to increase CBN content.

### BACKGROUND

The present disclosure relates to a method of enhancing the amount of Cannabinol (CBN) in Cannabis oil extracts.

Cannabis sativa including C. indica, C. ruderalis and their various sub species, varieties, hybrids, and eco-bio types contain different quantities of about 90 Phyto-cannabinoids. Among these are tetrahydrocannabinol (“THC”), Cannabidiol (“CBD”), Cannabinol (“CBN”), Cannabigerol (“CBG”), Cannabichromene (“CBC”) and tetrahydrocannabivarin (“THCV”, “THV”). Of these, THC and CBD are the most dominant and well-studied cannabinoids. In all cases, the Phyto cannabinoids exist as the acidic forms in the plant which, upon ageing of the plant and processing after harvest, get decarboxylated and converted to the nonacidic biologically active forms.

Thus, for example, both THC and CBD exist initially in their acid forms as THCA and CBDA and these get converted to THC and CBD by THCA synthase and CBD synthase, respectively, into the biologically active THC and CBD molecules.

### SUMMARY

A method of increasing Cannabidiol content in a Cannabis oil includes processing plant parts from one or more cannabis plants to create processed plant parts, extracting a Cannabis oil from the processed plant parts, and refining the Cannabis oil to increase CBN content.

The embodiments, examples, and alternatives of the preceding paragraph, the claims, or the following description and drawings, including any of their various aspects or respective individual features, may be taken independently or in any combination. Features described in connection with one embodiment are applicable to all embodiments, unless such features are incompatible.

### DETAILED DESCRIPTION

The present disclosure describes a method of increasing CBN content in a Cannabis oil. According to one example, the method includes processing plant parts from one or more cannabis plants to create processed plant parts, extracting a cannabis oil from the processed plant parts, and refining the Cannabis oil to increase CBN content. In one example, refining the Cannabis oil can also include converting THC in the Cannabis oil to CBN through oxidation.

In one example, processing the plant parts includes drying or freezing the plant parts in dry ice to create prepared plant parts. The prepared plant parts are then pulverized to create pulverized plant powders. The pulverized plant powders are suspended in a buffer containing 1 mg/ml each of pectinase and cellulases enzymes and incubated over night at 37° C. (99° F.) in containers placed on a shaker platform. However, the concentration of pectinase and cellulase may vary as needed in order to ensure optimal digestion of cell walls in the pulverized plant parts.

The containers are gently shaken on the shaker platform to distribute the contents thereby allowing the Cannabis plant cell walls in the pulverized plant powders to be digested to release the contents therein and produce digested extracts. In another example, the pulverized plat powders are incubated from 6 to 12 hours at a temperature between 35° C. (95° F.) and 39° C. (102° F.) to create the digested extracts.

The digested extracts are heated, such as with steam heat, to evaporate the aqueous phase leaving solids and Cannabis oil behind. The steam heat also converts any existing THC-A in the digested plant parts to THC. The material after steaming is extracted with 100 percent ethanol to dissolve pigments and all the cannabis oil to create an ethanol extract. The ethanol extract is filtered through a 100-mesh filter to remove the solids or cell debris. The ethanol extract free of solids is then filtered through a bed of charcoal to remove the pigments to create an ethanol filtrate. The ethanol filtrate contains the Cannabis oil and ethanol.

The ethanol filtrate is heated to evaporate the ethanol leaving behind the Cannabis oil. In an alternative example, the ethanol extract is subjected to further extraction procedures by super critical CO2 extraction. The oil fraction of the Cannabis oil is then collected.

The extracted Cannabis oil is then diluted with oil, such as coconut oil, olive oil, or hemp oil, to lighten and thin the Cannabis oil, which is otherwise relatively thick. Bubbling oxygen and ozone through the Cannabis oil while exposing the Cannabis oil to UV light for a period ranging between 1 and 24 hours converts as much of THC as possible to CBN. Some examples include bubbling and UV exposure for periods exceeding 24 hours. The timing of exposure to UV light and oxygen-ozone bubbling depends on the source plant from which the Cannabis oil is extracted, since different varieties of Cannabis have different profiles of cannabinoids. For example, some varieties of the Cannabis plants have larger percentages of THC compared to others and can benefit from longer exposures to convert the THC to CBN. The Cannabis oil with the THC converted to CBN can also be made into at least one of a water soluble form or a liposomal product.

After such processing, the Cannabis oil may be analyzed by Chromatography and mass spectrometry to determine levels of different cannabinoids and especially CBN. Some examples include a further purification step involving preparative column chromatography to obtain pure CBN. In some implementations, older Cannabis plants are used as starting material since they contain more CBN than younger plants. Younger plants indicate plants that are just ready to harvest, such as when trichomes turn a sticky golden brownish to dark, and older plants indicate a time period after this transition. In at least one example technique, the Cannabis oil is prepared from both male and female plants.

After the Cannabis oil is processed as described above, the oil can be labeled and stored for medical prescription use as oils, tinctures, tonics, and beverages. Alternatively, the oil is used in blends for approved medical purposes. Additionally, the Cannabis oil can be blended with other cannabinoids, cannabis terpenes, terpenes from other plant sources, flavanoids, L-theanine, various antioxidants from plants, flavoring agents, extracts from black pepper as bioavailability agents and other phytochemicals in order to formulate supplements that may be used to treat many different medical conditions in humans and animals.

Furthermore, any blend of Cannabis oil described in this disclosure may be imbibed with any other beverages pr therapy applications. Also, the blends may be used for medical conditions, such as sleep disorders, pain, sedation, inflammation, bone loss and osteoporosis, seizures, infections, appetite stimulation, glaucoma, hormone treatment and cancer.

Although the different non-limiting examples are illustrated as having specific components, the examples of this disclosure are not limited to those particular combinations. It is possible to use some of the components or features from any of the non-limiting examples in combination with features or components from any of the other non-limiting examples.

It should be understood that like reference numerals identify corresponding or similar elements throughout the several drawings. It should also be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claim should be studied to determine the true scope and content of this disclosure.

What is claimed is:

1. A method of increasing CBN content in a Cannabis oil, comprising:
processing plant parts from one or more cannabis plants to create processed plant parts;
extracting a Cannabis oil from the processed plant parts; and
refining the Cannabis oil to increase CBN content.

2. The method of claim 1, wherein the one or more cannabis plants include both male and female cannabis plants.

3. The method of claim 1, wherein the processing comprises:
at least one of drying or freezing plant tissues from the plant parts to create prepared plant parts;
pulverizing the prepared plant parts to create pulverized plant powders; and
incubating the pulverized plant powders.

4. The method of claim 3, wherein processing comprises:
suspending the pulverized plant powders in a buffer solution prior to incubating, wherein a concentration of pectinase and cellulases enzymes in the buffer solution is varied to ensure optimal digestion of cell walls in the pulverized plant parts.

5. The method of claim 4, wherein the buffer solution includes a concentration of 1 mg/ml each of pectinase and cellulases enzymes.

6. The method of claim 3, wherein the incubating comprises:
heating the pulverized plant parts to a temperature of between 35 and 39° C. for a period of between 6 to 12 hours; and
shaking the pulverized plant parts on a shaker platform while incubating to distribute the contents of the pulverized plant parts to create digested extracts.

7. The method of claim 6, wherein heating the pulverized plant parts when incubating includes heating to a temperature of 37° C.

8. The method of claim 6, comprising:
steam heating the digested extracts to evaporate an aqueous phase from the digested extracts to create heated extracts comprised of solids and the Cannabis oil.

9. The method of claim 8, wherein the steam heating converts THC-A in the digested plant parts into THC.

10. The method of claim 8, comprising:
dissolving the heated plant parts with 100 percent ethanol to dissolve pigments and the Cannabis oil to create an ethanol extract.

11. The method of claim 10, comprising:
filtering the ethanol extract through a 100-mesh filter to remove the solids and filtering through a bed of charcoal to remove the pigments to create an ethanol filtrate containing the ethanol with the Cannabis oil.

12. The method of claim 11, wherein refining comprises:
heating the ethanol filtrate containing the Cannabis oil and ethanol to evaporate the ethanol and leave behind the Cannabis oil.

13. The method of claim 12, comprising:
subjecting the ethanol filtrate to super critical CO2 extraction.

14. The method of claim 12, comprising:
diluting the Cannabis oil with at least one of coconut oil, olive oil, or hemp oil, or any other oil to lighten and thin the thickness of the Cannabis oil.

15. The method of claim 14, comprising:
bubbling Oxygen and ozone through the Cannabis oil while exposing the Cannabinoid oil to UV light for between 1 and 24 hours or longer to convert THC to CBN.

16. The method of claim 15, comprising:
purifying the Cannabis oil with preparative column chromatography to obtain pure CBN.

17. The method of claim 16, wherein the Cannabis oil is labeled and stored for medical prescription use as oils, tinctures, tonics, and beverages, or wherein the oil is used in blends for approved bio-medical purposes.

18. The method of claim 15, comprising blending the Cannabis oil with other cannabinoids, cannabis terpenes, terpenes from other plant sources, flavanoids, L-theanine, various antioxidants from plants, flavoring agents, extracts from black pepper as bioavailability agents and other phytochemicals in order to formulate supplements that may be used to treat many different medical conditions in humans and animals.

19. The method of claim 15, wherein the Cannabis oil with the THC converted to CBN is made into at least one of a water soluble form or a liposomal product.

20. The method of claim 1, comprising:
utilizing older Cannabis plants that contain more CBN than younger plants for the plant parts.

21. The method of claim 1, wherein refining the Cannabis oil includes converting THC in the Cannabis oil to CBN by oxidation.
